(19) United States
(12) Patent Application Publication
(10) Pub. No.: US 2020/0105415 A1
(43) Pub. Date: Apr. 2, 2020

(54) CONVERSATION GENERATION FOR DETAILING SYMPTOMS

(71) Applicant: INTERNATIONAL BUSINESS MACHINES CORPORATION, Armonk, NY (US)

(72) Inventors: Qi Cheng Li, Beijing (CN); Ya Bin Dang, Beijing (CN); Jian Wang, Beijing (CN); Li Jun Mei, Beijing (CN); Xin Zhou, Beijing (CN)

(21) Appl. No.: 16/144,602

(22) Filed: Sep. 27, 2018

(51) Int. Cl. G16H 50/30; G16H 10/20; G06F 17/28; G10L 15/00; G10L 13/00

(52) U.S. Cl. CPC G16H 50/30; G16H 10/20; G10L 13/00; G10L 15/00; G06F 17/28

(57) ABSTRACT

A computer-implemented method is provided for medical conversation generation. The method includes receiving, by a processor device, clinical findings for a patient. The method further includes calculating, by the processor device based on the clinical findings, (i) a suspected disease and (ii) symptoms for inquiry with (iii) a symptom significance for each of the symptoms. The method also includes decomposing, by the processor device, the symptoms from a multi-dimensional abstraction. The method additionally includes selecting, by the processor device, an inquiry strategy based on the symptom significance calculated for each of the symptoms. The method further includes calculating, by the processor device based on a decomposition of the symptoms and the inquiry strategy, a combination benefit/cost and evaluating the combination benefit/cost to provide an optimized combination result. The method also includes generating, by the processor device, an acoustic-based inquiry suite for the patient based on the optimized combination result.

### BACKGROUND

### Technical Field

The present invention generally relates to medical applications, and more particularly to conversation generation for detailing symptoms.

### Description of the Related Art

The clinical diagnosis and inquiry are key tasks performed by general practitioners. Most conventional systems and approaches focus on the diagnosis task, which attempts to identify the suspected diseases based on available findings. However, few of the conventional systems and approaches address the inquiry task in an efficient way, and usually involve apply simply rules to traverse possible symptoms, which is tedious and time-consuming. Hence, there is a need for an improved way to perform the inquiry task.

### SUMMARY

According to an aspect of the present invention, a computer-implemented method is provided for medical conversation generation. The method includes receiving, by a processor device, clinical findings for a patient. The method further includes calculating, by the processor device based on the clinical findings, (i) a suspected disease and (ii) symptoms for inquiry with (iii) a symptom significance for each of the symptoms. The method also includes decomposing, by the processor device, the symptoms from a multi-dimensional abstraction. The method additionally includes selecting, by the processor device, an inquiry strategy based on the symptom significance calculated for each of the symptoms. The method further includes calculating, by the processor device based on a decomposition of the symptoms and the inquiry strategy, a combination benefit/cost and evaluating the combination benefit/cost to provide an optimized combination result. The method also includes generating, by the processor device, an acoustic-based inquiry suite for the patient based on the optimized combination result.

According to another aspect of the present invention, a computer program product is provided for medical conversation generation. The computer program product includes a non-transitory computer readable storage medium having program instructions embodied therewith. The program instructions are executable by a computer to cause the computer to perform a method. The method includes receiving, by a processor device of the computer, clinical findings for a patient. The method further includes calculating, by the processor device based on the clinical findings, (i) a suspected disease and (ii) symptoms for inquiry with (iii) a symptom significance for each of the symptoms. The method also includes decomposing, by the processor device, the symptoms from a multi-dimensional abstraction. The method additionally includes selecting, by the processor device, an inquiry strategy based on the symptom significance calculated for each of the symptoms. The method further includes calculating, by the processor device based on a decomposition of the symptoms and the inquiry strategy, a combination benefit/cost and evaluating the combination benefit/cost to provide an optimized combination result. The method also includes generating, by the processor device, an acoustic-based inquiry suite for the patient based on the optimized combination result.

According to yet another aspect of the present invention, a computer processing system is provided for medical conversation generation. The computer processing system includes a memory for storing program code. The computer processing system further includes a processor device for running the program code to receive clinical findings for a patient. The processor further runs the program code to calculate, based on the clinical findings, (i) a suspected disease and (ii) symptoms for inquiry with (iii) a symptom significance for each of the symptoms. The processor also runs the program code to decompose the symptoms from a multi-dimensional abstraction. The processor additionally runs the program code to select an inquiry strategy based on the symptom significance calculated for each of the symptoms. The processor further runs the program code to calculate, based on a decomposition of the symptoms and the inquiry strategy, a combination benefit/cost and evaluating the combination benefit/cost to provide an optimized combination result. The processor also runs the program code to generate an acoustic-based inquiry suite for the patient based on the optimized combination result.

These and other features and advantages will become apparent from the following detailed description of illustrative embodiments thereof, which is to be read in connection with the accompanying drawings.

### BRIEF DESCRIPTION OF THE DRAWINGS

The following description will provide details of preferred embodiments with reference to the following figures wherein:

FIG. 1 is a block diagram showing an exemplary processing system to which the present invention may be applied, in accordance with an embodiment of the present invention;

FIG. 2 is a block diagram showing an exemplary system for conversation generation for detailing symptoms, in accordance with an embodiment of the present invention;

FIG. 3 is a flow diagram showing an exemplary method for conversation generation for detailing symptoms, in accordance with an embodiment of the present invention;

FIG. 4 is a block diagram showing an illustrative cloud computing environment having one or more cloud computing nodes with which local computing devices used by cloud consumers communicate, in accordance with an embodiment of the present invention; and

FIG. 5 is a block diagram showing a set of functional abstraction layers provided by a cloud computing environment, in accordance with an embodiment of the present invention.

### DETAILED DESCRIPTION

The present invention is directed to conversation generation for detailing medical symptoms. In an embodiment, the present invention is directed to the inquiry portion of the two-part approach of clinical inquiry and clinical diagnosis. To that end, the present invention provides a comprehensive and dynamic approach to inquiry suggestion in order to suggest an optimized inquiry that leads to a more accurate diagnosis. These and other advantages of the present invention are described in further detail hereinbelow.

In an embodiment, the present invention generates an inquiry suggestion based on the required symptoms (to implicate that suggestion), and also a comprehensive combination with symptom conditional probability, confidence of suspected disease, and common aspects of their abstraction.

The present invention automates what has typically been a strictly human-based and tedious, rigid, and time-consuming approach. Moreover, the present invention enhances conventional approaches using a more comprehensive and detailed outcome while being more efficient than typical conventional approaches. For example, the present invention is easier to implement and more comprehensive than static list-based approaches. Additionally, the present invention provides a better dynamic conversational experience that dynamic user input approaches. In this way, an inquiry suggestion can be generated more quickly, efficiently, comprehensively, dynamically, and so forth over conventional approaches.

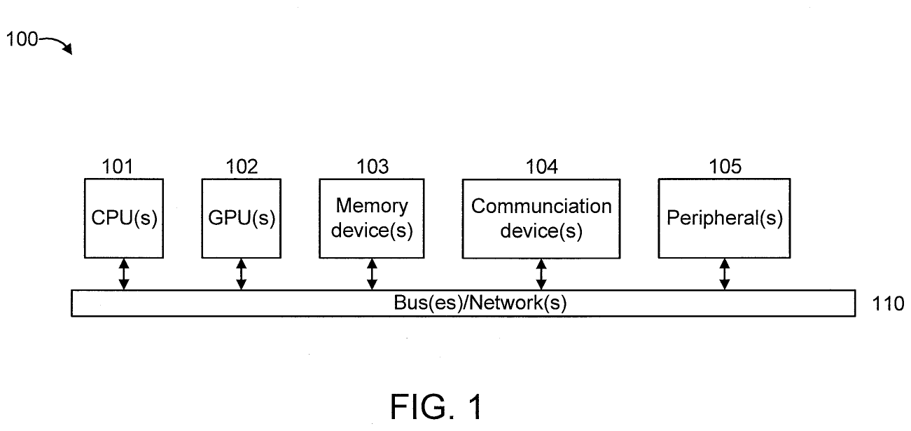

FIG. 1 is a block diagram showing an exemplary processing system 100 to which the present invention may be applied, in accordance with an embodiment of the present invention. The processing system 100 includes a set of processing units (e.g., CPUs) 101, a set of GPUs 102, a set of memory devices 103, a set of communication devices 104, and set of peripherals 105. The CPUs 101 can be single or multi-core CPUs. The GPUs 102 can be single or multi-core GPUs. The one or more memory devices 103 can include caches, RAMs, ROMs, and other memories (flash, optical, magnetic, etc.). The communication devices 104 can include wireless and/or wired communication devices (e.g., network (e.g., WIFI, etc.) adapters, etc.). The peripherals 105 can include a display device, a user input device, a printer, and so forth. Elements of processing system 100 are connected by one or more buses or networks (collectively denoted by the figure reference numeral 110).

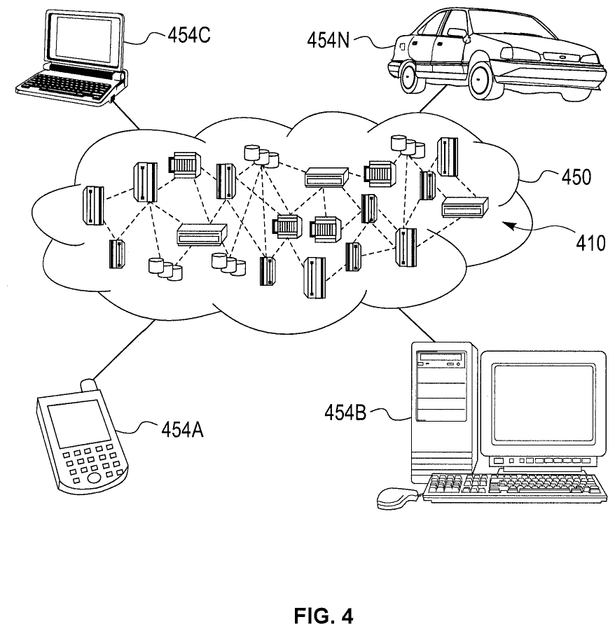

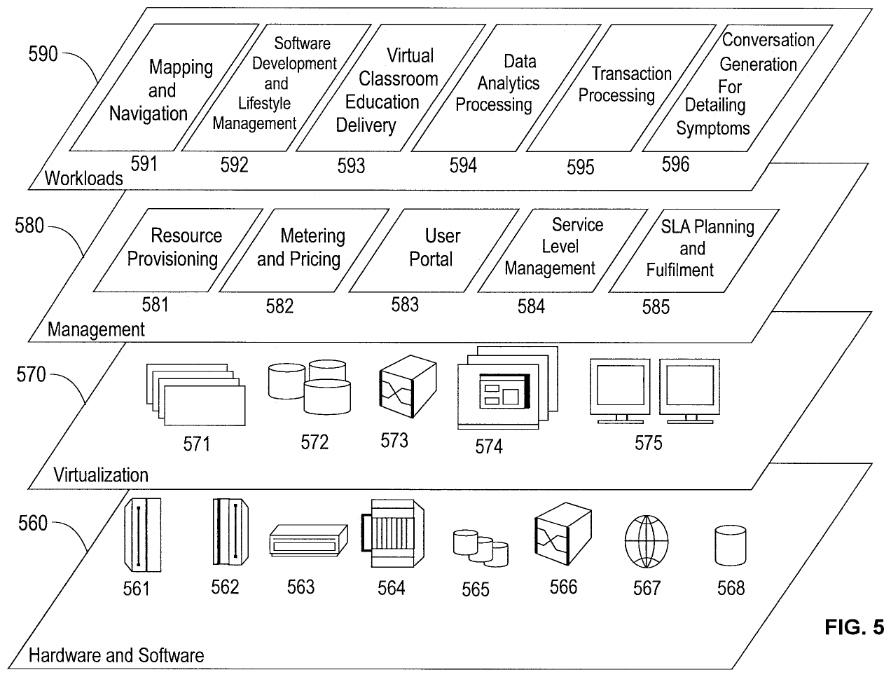

Of course, the processing system 100 may also include other elements (not shown), as readily contemplated by one of skill in the art, as well as omit certain elements. For example, various other input devices and/or output devices can be included in processing system 100, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. Further, in another embodiment, a cloud configuration can be used (e.g., see FIGS. 4-5). For example, system 100 can represent at least a portion of a node in a cloud computing environment. These and other variations of the processing system 100 are readily contemplated by one of ordinary skill in the art given the teachings of the present invention provided herein.

Moreover, it is to be appreciated that various figures as described below with respect to various elements and steps relating to the present invention that may be implemented, in whole or in part, by one or more of the elements of system 100.

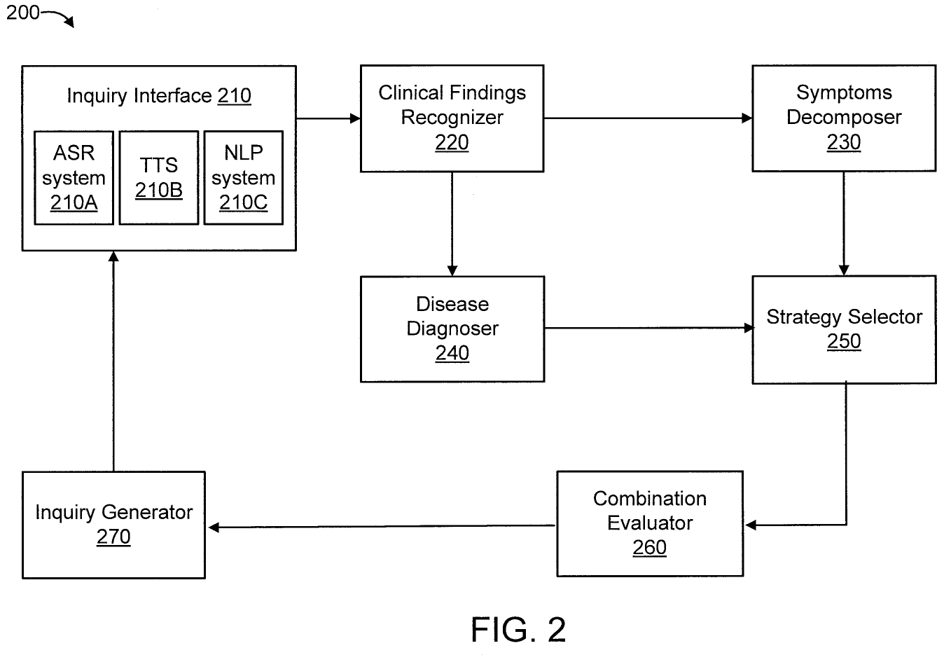

FIG. 2 is a block diagram showing an exemplary system 200 for conversation generation for detailing symptoms, in accordance with an embodiment of the present invention.

The system 200 include an inquiry interface 210, a clinical findings recognizer 220, a symptoms decomposer 230, a disease diagnoser 240, a strategy selector 250, a combination evaluator 260, and an inquiry generator 270.

The inquiry interface 210 provides one or more user interfaces for conversation generation for detailing symptoms. The inquiry interface 210 can include one or more dashboards. In an embodiment, the dashboards can be organized based on disease (where each dashboard pertains to one of a set of multiple diseases, etc.) or some other characterization. The inquiry interface 210 can be used to input clinical findings and/or other information into the system 200 for use to ultimately generate an inquiry task. The inquiry interface 210 can provide the inquiry task to a user that is generated by the inquiry generator 270.

In an embodiment, the inquiry interface 210 includes an Automatic Speech Recognition (ASR) system 210A, a Text-To-Speech (TTS) system 210B, and a Natural Language Processing (NLP) system 210C for enabling a conversation style exchange between a user (e.g., a patient, a health care practitioner, etc.) and system 200. In an embodiment, the ASR system 210A is used to recognize utterances generated by a user, and the TTS system 210B and NLP system are used to generate speech uttered to a user in a natural language manner.

The clinical findings recognizer 220 receives clinical findings from the inquiry interface 210 and determines symptoms based on the clinical findings. In an embodiment, the clinical findings recognizer further determines a suspicion of one or more diseases based on the clinical findings.

The symptoms decomposer 230 receives a set(s) of symptoms (determined by the clinical findings recognizer 220) and decomposes the set(s) of symptoms into subsets of symptoms based on common abstraction aspects e.g., body part, symptom model, pathophysiology, and so forth.

The disease diagnoser 240 generates a disease diagnosis based on the symptoms (determined by the clinical findings recognizer 220).

The strategy selector 250 selects all strategies based on the subsets of symptoms (from the symptoms decomposer 230) and the disease diagnosis (from the disease diagnoser 240).

The combination evaluator 260 evaluates the costs of every selected strategy.

The inquiry generator 270 generates the inquiry task based on a combination evaluation based result (from the combination evaluator 260).

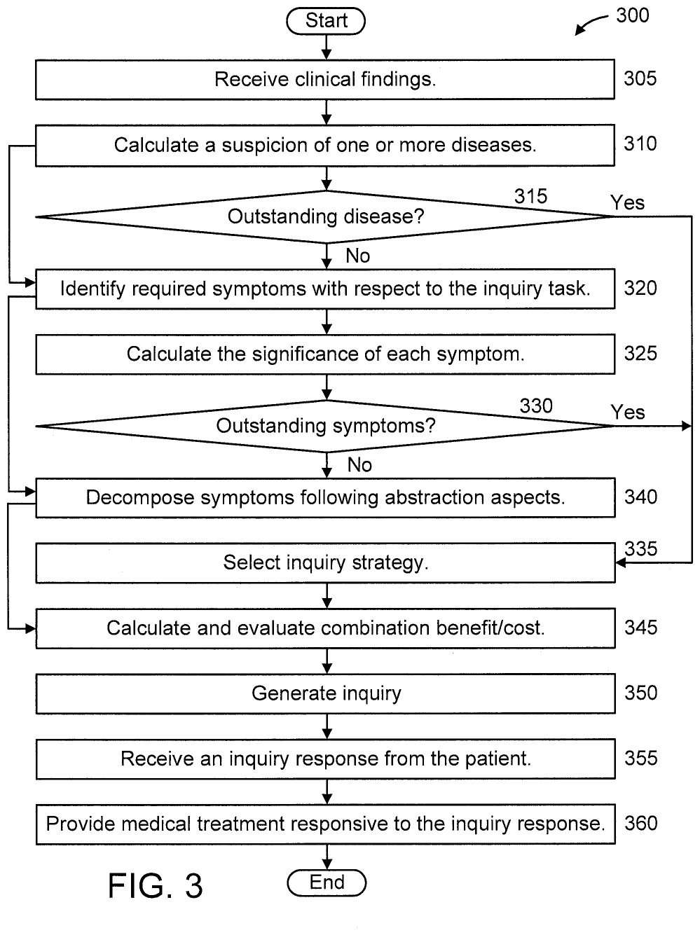

FIG. 3 is a flow diagram showing an exemplary method 300 for conversation generation for detailing symptoms, in accordance with an embodiment of the present invention.

At block 305, receive clinical findings. In an embodiment, block 305 can involve one or more transformation of the clinical findings from one state to another state. For example, clinical images can be converted into text and/or another format for use by the present invention. A text conversion process can apply labels to various aspects of the clinical findings, based on reference images and/or so forth.

At block 310, calculate a suspicion of one or more diseases.

At block 315, determine whether or not the suspicion is of an outstanding disease. If so, then proceed to block 335. Otherwise, proceed to block 320.

At block 320, identify required symptoms with respect to the inquiry task.

At block 325, calculate the significance of each symptom.

At block 330, determine whether or not the symptom is of an outstanding symptom. If so, then proceed to block 335. Otherwise, proceed to block 340.

At block 335, select inquiry strategy.

At block 340, decompose symptoms following abstraction aspects.

At block 345, calculate and evaluate combination benefit/cost.

At block 350, generate inquiry using NLP to commence a natural language conversation with the patient.

At block 355, receive an inquiry response from the patient.

At block 360, provide medical treatment responsive to the inquiry response. The medical treatment can involve an automated injection, an automated blood pressure measurement, configuration of parameters of an imaging machine to expose the patient for further information acquisition, and so forth. These and other actions relating to providing medical treatment are readily contemplated by one of ordinary skill in the art, given the teachings of the present invention provided herein, while maintaining the spirit of the present invention.

Further regarding block 335, the inquiry strategy selection in a multi-dimensional approach can involve, but are not limited to, one or more of the following: (i) combination versus discrimination; (ii) cross-diseases combination; and (iii) symbiosis combination.

Further regarding block 340, common abstraction aspects in a multi-dimensional approach can involve, but are not limited to, one or more of the following: body part; symptoms model; and pathophysiology.

In an embodiment, block 340 can involve the construction or use of a knowledge graph, which is navigated based on the patient's claims (e.g., clinical findings and symptoms) to find related symptoms for use to generate a natural language conversation with the patient to clarify the symptom details. In an embodiment, the knowledge graph can be constructed as a symptoms forest, such that the tress in the forest are navigated (traversed) to finds the corresponding symptoms, differences between sub-symptoms, and so forth. In an embodiment, the symptoms tree can be constructed so that each tree corresponds to a particular disease. Of course, other construction arrangements can also be used. An utterance for different attributes of sub-symptoms can be extracted from the tree for use in conversing with the patient during the inquiry task.

Further regarding block 350, an exemplary combination value calculation can involve the following: minimize the cost C, where C(S₀, S₁, . . . , Sₙ₋₁)=(n+1)·P(∪ᵢⁿ Sᵢ)+P(∩ᵢⁿ S̅ᵢ), where S denotes the symptom would appear, n denotes the count of current symptom set, P denotes the function to calculate probability, and S̅ denotes the symptom would not appear.

Also, further regarding block 350, the inquiry can be provided to the user by the TTS 210B and the NLP system 210C in a conversational manner.

Additionally, further regarding block 35, exemplary inquiry generations can include, but are not limited to the following:

(a) “Do you have pharynx discomfort?”->“pharyngoxerosis”, “throat itch”, “throat pain”
(b) “Is there blood in the stool?”->“Blood on the back end”, “Blood on the front end”
(c) “Do you have colic in the abdomen?”->“upleft”, “upright”, . . .
(d) “Itching of the nasal cavity” or “Sneezing” or “Runny nose” (under “Rhinallergosis”)

In an embodiment, each of multiple instances of system 200 can be implemented by each of multiple nodes in a distributed cloud computing system. An overall machine learning approach can be applied across the nodes in order to improve the results at each node by increasing the knowledge base and resultant mappings (between user inputs and symptoms/etc.) of the system 200 in order to generate an optimized inquiry suggestion. Exemplary cloud implementations are described below with respect to FIGS. 4-5. These and other configurations of the present invention are readily determined by one of ordinary skill in the art, given the teachings of the present invention provided herein, while maintaining the spirit of the present invention.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 4, illustrative cloud computing environment 450 is depicted. As shown, cloud computing environment 450 includes one or more cloud computing nodes 410 with which local computing devices used by cloud consumers, such as, for example, personal digital assistant (PDA) or cellular telephone 454A, desktop computer 454B, laptop computer 454C, and/or automobile computer system 4554N may communicate. Nodes 410 may communicate with one another. They may be grouped (not shown) physically or virtually, in one or more networks, such as Private, Community, Public, or Hybrid clouds as described hereinabove, or a combination thereof. This allows cloud computing environment 450 to offer infrastructure, platforms and/or software as services for which a cloud consumer does not need to maintain resources on a local computing device. It is understood that the types of computing devices 454A-N shown in FIG. 4 are intended to be illustrative only and that computing nodes 410 and cloud computing environment 450 can communicate with any type of computerized device over any type of network and/or network addressable connection (e.g., using a web browser).

Referring now to FIG. 5, a set of functional abstraction layers provided by cloud computing environment 450 (FIG. 4) is shown. It should be understood in advance that the components, layers, and functions shown in FIG. 5 are intended to be illustrative only and embodiments of the invention are not limited thereto. As depicted, the following layers and corresponding functions are provided:

Hardware and software layer 560 includes hardware and software components. Examples of hardware components include: mainframes 561; RISC (Reduced Instruction Set Computer) architecture based servers 562; servers 563; blade servers 564; storage devices 565; and networks and networking components 566. In some embodiments, software components include network application server software 567 and database software 568.

Virtualization layer 570 provides an abstraction layer from which the following examples of virtual entities may be provided: virtual servers 571; virtual storage 572; virtual networks 573, including virtual private networks; virtual applications and operating systems 574; and virtual clients 575.

In one example, management layer 580 may provide the functions described below. Resource provisioning 581 provides dynamic procurement of computing resources and other resources that are utilized to perform tasks within the cloud computing environment. Metering and Pricing 582 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 583 provides access to the cloud computing environment for consumers and system administrators. Service level management 584 provides cloud computing resource allocation and management such that required service levels are met. Service Level Agreement (SLA) planning and fulfillment 585 provide pre-arrangement for, and procurement of, cloud computing resources for which a future requirement is anticipated in accordance with an SLA.

Workloads layer 590 provides examples of functionality for which the cloud computing environment may be utilized. Examples of workloads and functions which may be provided from this layer include: mapping and navigation 591; software development and lifecycle management 592; virtual classroom education delivery 593; data analytics processing 594; transaction processing 595; and role-oriented risk checking in contract review based on deep semantic association analysis 596.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as SMALLTALK, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

Having described preferred embodiments of a system and method (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

What is claimed is:

1. A computer-implemented method for medical conversation generation, comprising:
receiving, by a processor device, clinical findings for a patient;
calculating, by the processor device based on the clinical findings, (i) a suspected disease and (ii) symptoms for inquiry with (iii) a symptom significance for each of the symptoms;
decomposing, by the processor device, the symptoms from a multi-dimensional abstraction;
selecting, by the processor device, an inquiry strategy based on the symptom significance calculated for each of the symptoms;
calculating, by the processor device based on a decomposition of the symptoms and the inquiry strategy, a combination benefit/cost and evaluating the combination benefit/cost to provide an optimized combination result; and
generating, by the processor device, an acoustic-based inquiry suite for the patient based on the optimized combination result.

2. The computer-implemented method of claim 1, wherein the inquiry strategy is selected from the group consisting of (a) combination versus discrimination; (b) cross-diseases combination; and (c) symbiosis combination.

3. The computer-implemented method of claim 1, wherein the multi-dimensional abstraction comprises element representative of various body parts with corresponding diseases and the symptoms of the corresponding diseases.

4. The computer-implemented method of claim 1, wherein the multi-dimensional abstraction comprises a symptoms model.

5. The computer-implemented method of claim 1, further comprising converting to the clinical findings from an image-based format to a text-based format.

6. The computer-implemented method of claim 1, further comprising forming a symptoms forest with each of trees of the symptoms forest corresponding to a respective different one of different diseases, and tree branches of the trees corresponding to the symptoms of the different diseases.

7. The computer-implemented method of claim 1, wherein said generating step generates the acoustic-based inquiry suite using a natural language processing system and a text-to-speech system.

8. The computer-implemented method of claim 1, wherein said generating step comprises responding to patient replies to the inquiry suite in a conversational manner, wherein said responding step is performed to specifically obtain additional symptom details from the patient to enhance a diagnosis for the patient.

9. A computer program product for medical conversation generation, the computer program product comprising a non-transitory computer readable storage medium having program instructions embodied therewith, the program instructions executable by a computer to cause the computer to perform a method comprising:
receiving, by a processor device of the computer, clinical findings for a patient;
calculating, by the processor device based on the clinical findings, (i) a suspected disease and (ii) symptoms for inquiry with (iii) a symptom significance for each of the symptoms;
decomposing, by the processor device, the symptoms from a multi-dimensional abstraction;
selecting, by the processor device, an inquiry strategy based on the symptom significance calculated for each of the symptoms;
calculating, by the processor device based on a decomposition of the symptoms and the inquiry strategy, a combination benefit/cost and evaluating the combination benefit/cost to provide an optimized combination result; and
generating, by the processor device, an acoustic-based inquiry suite for the patient based on the optimized combination result.

10. The computer program product of claim 9, wherein the inquiry strategy is selected from the group consisting of (a) combination versus discrimination; (b) cross-diseases combination; and (c) symbiosis combination.

11. The computer program product of claim 9, wherein the multi-dimensional abstraction comprises element representative of various body parts with corresponding diseases and the symptoms of the corresponding diseases.

12. The computer program product of claim 9, wherein the multi-dimensional abstraction comprises a symptoms model.

13. The computer program product of claim 9, wherein the method further comprises converting to the clinical findings from an image-based format to a text-based format.

14. The computer program product of claim 9, wherein the method further comprises forming a symptoms forest with each of trees of the symptoms forest corresponding to a respective different one of different diseases, and tree branches of the trees corresponding to the symptoms of the different diseases.

15. The computer program product of claim 9, wherein said generating step generates the acoustic-based inquiry suite using a natural language processing system and a text-to-speech system implemented by the computer.

16. The computer program product of claim 9, wherein said generating step comprises responding to patient replies to the inquiry suite in a conversational manner, wherein said responding step is performed to specifically obtain additional symptom details from the patient to enhance a diagnosis for the patient.

17. A computer processing system for medical conversation generation, comprising:
a memory for storing program code; and
a processor device for running the program code to
receive clinical findings for a patient;
calculate, based on the clinical findings, (i) a suspected disease and (ii) symptoms for inquiry with (iii) a symptom significance for each of the symptoms;
decompose the symptoms from a multi-dimensional abstraction;
select an inquiry strategy based on the symptom significance calculated for each of the symptoms;
calculate, based on a decomposition of the symptoms and the inquiry strategy, a combination benefit/cost and evaluating the combination benefit/cost to provide an optimized combination result; and
generate an acoustic-based inquiry suite for the patient based on the optimized combination result.

18. The computer processing system of claim 17, further comprising a natural language processing system and a text-to-speech system for enabling a conversational exchange between the user and the computer processing system directed to obtaining additional details on the symptoms of the patient.

19. The computer processing system of claim 17, further comprising an automatic speech recognition system for recognizing patient uttered replies to the inquiry suite.

20. The computer processing system of claim 17, wherein said processor device further runs the program code to form a symptoms forest with each of trees of the symptoms forest corresponding to a respective different one of different diseases, and tree branches of the trees corresponding to the symptoms of the different diseases.
